(19) United States
(12) Patent Application Publication
(10) Pub. No.: US 2006/0205752 A1
(43) Pub. Date: Sep. 14, 2006

(54) Stabilized hydrocodone pharmaceutical compositions with ethylenediaminetetraacetic acid

(76) Inventor: Keith Whitehead, Oldsmar, FL (US)

Correspondence Address: WYATT BARTON PRATT, 15 COLUMBIA AVENUE, HOPEWELL, NJ, 08525, US

(21) Appl. No.: 11/079,864

(22) Filed: Mar. 14, 2005

(51) Int. Cl. A61K 31/485; A61K 31/195

(52) U.S. Cl. 514/282; 514/566

(57) ABSTRACT

Hydrocodone pharmaceutical formulations are stabilized with a stabilizing effective amount of ethylenediaminetetraacetic compound.

### BACKGROUND OF THE INVENTION

1. Field of the Invention

The present invention pertains to pharmaceutical compositions of hydrocodone stabilized with ethylenediaminetetraacetic acid.

2. Brief Description of the Related Art

Hydrocodone is chemically known as 4,5-epoxy-3-methoxy-17-methyl-morphinan-6-one. The synthesis of hydrocodone and its pharmaceutically acceptable acid addition salts are described in U.S. Pat. No. 2,715,626 issued to Pfister et al, and in the Merck Index, 11th Edition, page 757, entry 4708 (1989). Hydrocodone is a narcotic antitussive and analgesic. Hydrocodone presents stability problems in pharmaceutical formulations.

There is a need in the art to provide improved stability of hydrocodone pharmaceutical formulations. The present invention addresses this and other needs.

### SUMMARY OF THE INVENTION

The present invention includes a stabilized hydrocodone pharmaceutical formulation having a pharmaceutically effective amount of hydrocodone with a stabilizing effective amount of ethylenediaminetetraacetic compound for stabilizing the hydrocodone. In preferred embodiments, the hydrocodone pharmaceutical formulation includes a pharmaceutically effective amount of at least one additional active agent such as acetaminophen. The hydrocodone formulations are preferably in liquid form such as a solution, and the solutions may be administered as a unit dosage, such as a unit dosage of 15 mL.

The present invention also includes a method for stabilizing a hydrocodone formulation comprising the steps of formulating a hydrocodone composition and, to the formulated hydrocodone composition, adding an effectively stabilizing amount of ethylenediaminetetraacetic compound. The method may include the addition of one or more additional active agents.

### DETAILED DESCRIPTION OF THE INVENTION

The present invention includes a hydrocodone pharmaceutical formulation stabilized with an ethylenediaminetetraacetic acid (EDTA), including one or more of its salts. It has been surprisingly found that hydrocodone is particularly well stabilized with the addition of EDTA. It has also been found that additional active agents may be included in the hydrocodone formulation while maintaining the improved stability of the hydrocodone.

When used herein, the terms “active agent”, “active pharmaceutical ingredient”, “pharmaceutical actives”, “API”, “active compound”, “therapeutic agent”, “therapeutic ingredient”, “therapeutic compound” and other like terms are used interchangeably and include salts and other pharmaceutical forms of the detailed compounds. The terms “additional active agent”, “second active agent” or “other pharmaceutical active” include those active agents in addition to the hydrocodone.

Hydrocodones of the present invention include the basic forms and pharmaceutically acceptable salts, such as phosphate, sulfate, hydrochloride and bitartrate, of the hydrocodone. Preparation of the hydrocodone is known in the art, such as that disclosed in U.S. Pat. No. 2,715,626 to Pfister et al., entitled “Process of Preparing Dihydrocodeinone”, the disclosure of which is herein incorporated by reference. One well-known chemically formula of a hydrocodone is 4,5α-epoxy-3-methoxy-17-methylmorphinan-6-one tartrate. Representative pharmaceutically acceptable hydrocodone include it salts, such as hydrocodone bitartrate, hydrocodone bitartrate hydrate, hydrocodone hydrochloride, hydrocodone p-toluenesulfonate, hydrocodone phosphate, hydrocodone thiosemicarbazone, hydrocodone sulfate, hydrocodone trifluoroacetate, hydrocodone hemipentahydrate, hydrocodone pentafluoropropionate, hydrocodone p-nitrophenylhydrazone, hydrocodone o-methyloxime, hydrocodone semicarbazone, hydrocodone hydrobromide, hydrocodone mucate, hydrocodone oleate, hydrocodone phosphate dibasic, hydrocodone phosphate monobasic, hydrocodone inorganic salt, hydrocodone organic salt, hydrocodone acetate trihydrate, hydrocodone bis(heptafuorobutyrate), hydrocodone bis(methylcarbamate), hydrocodone bis(pentafluoropropionate), hydrocodone bis(pyridine carboxylate), hydrocodone bis(trifluoroacetate), hydrocodone chlorhydrate, hydrocodone sulfate pentahydrate and combinations thereof. Preferably, the hydrocodone is present as its bitartrate salt.

Preferably, the hydrocodone pharmaceutical formulation comprises an oral liquid composition, such as emulsions, solutions, suspensions, syrups, elixirs and combinations thereof. In a more preferred embodiment, the formulation includes a solution. The hydrocodone is present in the formulation in a pharmaceutically effective amount for administration to an individual. Representative amounts of hydrocodone include from about 2 mg to about 12 mg, and more preferably from about 5 mg to about 10 mg per dosage, such as in a liquid dosage of 15 mL. More preferably the hydrocodone is present in an amount of about 7.5 mg per tablet or per 15 mL of liquid. The solutions of the present invention are suitable for incorporating a variety of components for oral administration in an aqueous liquid in addition to the hydrocodone API.

Ethylenediaminetetraacetic acid (EDTA; edetate disodium), chemically known as (HO₂CCH₂)₂NCH₂CH₂N(CH₂CO₂H)₂, is commercially available both as the free acid and as various salts. Considering the widespread use of EDTA and the volume of its manufacture, both its manufacture and its commercial use, as well as disclosures such as U.S. Pat. No. 2,407,645 to Bersworth, entitled “Aliphatic Polycarboxylic Amino Acids and Process of Making Them”, EDTA is well known to persons skilled in the art. Representative examples of the EDTA for inclusion in the present invention include, for example, di-potassium salt, di-sodium salt, calcium/di-sodium salt, sodium salt, tri-sodium salt, tetrasodium salt and the like, and combinations thereof.

The EDTA is included in the formulation in amounts that effectively stabilize the hydrocodone. Stabilization of the hydrocodone includes the preservation of the hydrocodone and maintaining the hydrocodeone as a pharmaceuctically effective active agent for the purpose of its consumption. Stabilization of the hydrocodone by the EDTA as taught herein provides, for example, increase stability of the hydrocodone from 3 months (without the use of the hydrocodone) to 6 months (with the addition of EDTA as a stabilizer). Representative amounts of ethylenediaminetetraacetic acid for effective stabilization of the hydrocodone include, for example, from about 0.01% w/v to about 0.2% w/v, more preferably in an amount of from about 0.03% w/v to about 0.1% w/v, and most preferably about 0.05% w/v.

In addition to the hydrocodone API and EDTA in the solution of the present invention, the solutions may include other pharmaceutical actives in combination with the hydrocodone. The dosage forms of the present invention may further include one or more additional active agents (“second active agent”) which may or may not act synergistically with the hydrocodone of the present invention. Other pharmaceutical actives suitable for use in the present invention include, for example without limitation, such additional active agents as acebutolol, acetaminophen, acetylcysteine, acetylsalicylic acid, acyclovir, alfacalcidol, allantoin, allopurinol, alprazolam, ambroxol, amikacin, amiloride, aminoacetic acid, amiodarone, amitriptyline, amlodipine, amoxicillin, ampicillin, ascorbic acid, aspartame, astemizole, atenolol, beclomethasone, benserazide, benzalkoniumhydrochloride, benzocaine, benzoic acid, betamethasone, bezafibrate, biotin, biperiden, bisoprolol, bromazepam, bromhexine, bromocriptine, budesonide, bufexamac, buflomedil, buspirone, caffeine, camphor, captopril, carbamazepine, carbidopa, carboplatin, cefachlor, cefadroxil, cefalexin, cefazolin, cefixime, cefotaxime, ceftazidime, ceftriaxone, cefuroxime, chloramphenicol, chlorhexidine, chlorpheniramine, chlortalidone, choline, cyclosporin, cilastatin, cimetidine, ciprofloxacin, cisapride, cisplatin, clarithromycin, clavulanic acid, clomipramine, clonazepam, clonidine, clotrimazole, codeine, cholestyramine, cromoglycic acid, cyanocobalamin, cyproterone, desogestrel, dexamethasone, dexpanthenol, dextromethorphan, dextropropoxiphene, diazepam, diclofenac, digoxin, dihydrocodeine, dihydroergotamine, dihydroergotoxin, diltiazem, diphenhydramine, dipyridamole, dipyrone, disopyramide, domperidone, dopamine, doxocyclin, enalapril, ephedrine, epinephrine, ergocalciferol, ergotamine, erythromycin, estradiol, ethinylestradiol, etoposide, Eucalyptus globulus, famotidine, felodipine, fenofibrate, fenoterol, fentanyl, flavin-mononucleotide, fluconazole, flunarizine, fluorouracil, fluoxetine, flurbiprofen, folinic acid, furosemide, gallopamil, gemfibrozil, gentamicin, Gingko biloba, glibenclamide, glipizide, clozapine, glycyrrhiza glabra, griseofulvin, guaifenesin, haloperidol, heparin, hyaluronic acid, hydrochlorothiazide, hydrocodone, hydrocortisone, hydromorphone, ipratropium-hydroxide, ibuprofen, imipenem, indomethacin, iohexol, iopamidol, isosorbide-dinitrate, isosorbide-mononitrate, isotretinoin, ketotifen, ketoconazole, ketoprofen, ketorolac, labetalol, lactulose, lecithin, levocamitine, levodopa, levoglutamide, levonorgestrel, levothyroxine, lidocaine, lipase, imipramine, lisinopril, loperamide, lorazepam, lovastatin, medroxyprogesterone, menthol, methotrexate, methyldopa, methylprednisolone, metoclopramide, metoprolol, miconazole, midazolam, minocycline, minoxidil, misoprostol, morphine, multivitamin mixtures or combinations and mineral salts, N-methylephedrine, naftidrofuryl, naproxen, neomycin, nicardipine, nicergoline, nicotinamide, nicotine, nicotinic acid, nifedipine, nimodipine, nitrazepam, nitrendipine, nizatidine, norethisterone, norfloxacin, norgestrel, nortriptyline, nystatin, ofloxacin, omeprazole, ondansetron, pancreatin, panthenol, pantothenic acid, paracetamol, penicillin G, penicillin V, pentoxifylline, phenobarbital, phenoxymethylpenicillin, phenylephrine, phenylpropanolamine, phenyloin, piroxicam, polymyxin B, povidone-iodine, pravastatin, prazepam, prazosin, prednisolone, prednisone, propafenone, propranolol, proxyphylline, pseudoephedrine, pyridoxine, quinidine, ramipril, ranitidine, reserpine, retinol, riboflavin, rifampicin, rutoside, saccharin, salbutamol, salcatonin, salicylic acid, selegiline, simvastatin, somatropin, sotalol, spironolactone, sucralfate, sulbactam, sulfamethoxazole, sulfasalazine, sulpiride, tamoxifen, tegafur, teprenone, terazosin, terbutaline, terfenadine, tetracycline, theophylline, thiamine, ticlopidine, timolol, tranexamic acid, tretinoin, triamcinolone-acetonide, triamterene, trimethoprim, troxerutin, uracil, valproic acid, vancomycin, verapamil, vitamin E, zidovudine and the like. Preferred among these active agents to be included in the hydrocodone formulation is acetaminophen.

Acetaminophen (APAP), chemically known as N-acetyl-para-aminophenol or N-(4-hydroxyphenyl)acetamide, is a well-known over-the-counter analgesic and anti-pyretic agent. Acetaminophen was first used in medicine in 1893, and is currently available as an over the counter drug product. Preparation of APAP is disclosed in such publications as U.S. Pat. No. 2,998,450 to Wilbert et al., entitled “Process of Preparing N-Acetyl-p-Amino Phenol” and U.S. Pat. No. 5,155,273 to Fritch et al., entitled “Production of Acetaminophen”. Considering the widespread use of APAP and the volume of its manufacture, both its manufacture and its use as an analgesic is well known to persons skilled in the art. Pharmaceutically effective amounts of acetaminophen include those amounts appropriate for pharmacological relief of symptoms or conditions of a person in need of medical or other like treatment, with representative amounts ranging from about 125 mg to about 750 mg per tablet or per 15 mL, more preferably from about 250 mg to about 750 mg, and most preferably in an amount of about 500 mg per 15 mL.

Unit dosages of the hydrocodone formulation include liquid combinations of hydrocodone with EDTA, optionally with at least a second active agent, having appropriate amounts of the stabilized hydrocodone for treatment. Representative unit dosages include hydrocodone present in an amount of about 7.5 mg, and the ethylenediaminetetraacetic compound present in an amount of about 0.05 w/v, per 15 mL of solution. When a second active agent is present in the formulation, that agent is also present in amounts appropriate for treatment, such as acetaminophen present in an amount of about 500 mg per 15 mL of solution.

The hydrocodone formulation is produced by formulating a hydrocodone composition, generally in an aqueous medium to create a dissolved hydrocodone in solution, adding an effectively stabilizing amount of ethylenediaminetetraacetic acid to this solution and formulating the hydrocodone/ethylenediaminetetraacetic acid solution into an appropriate solid or liquid form. Additional excipients, as described herein, are included in the formulation to prepare a pharmaceutically acceptable drug product. When a second, or more, active agents are included, such active agents may be further incorporated into the dissolved hydrocodone solution for dispersion therein. Preparation of the aqueous pharmaceutical composition of the present invention is accomplished using ingredients of a purity such that it is suitable for administration to patients. Generally, the pharmaceutical formulation contains at least one conventional pharmaceutical excipient in addition to the hydrocodone and EDTA.

As an oral pharmaceutical formulation, the present invention may include commonly used excipients useful for such administration. Representative excipients, such as that for an aqueous formulation include, for example, buffering agents, preservatives, viscosity enhancing agents, pH moderating agents, sweeteners, flavor and/or flavoring aids, etc.

The present invention may include suitable buffers (also referred to herein as “buffer salts” or “buffering agent”). As used herein, the term “buffers” is intended to mean a compound used to resist a change in pH upon dilution or addition of acid or alkali. Preferred buffer salts include, by way of example and without limitation, potassium dihydrogen orthophosphate, disodium hydrogen orthophosphate, citric acid and disodium hydrogen orthophosphate, potassium phosphate dibasic, sodium phosphate dibasic, potassium metaphosphate, potassium phosphate, monobasic sodium acetate and sodium citrate anhydrous and dehydrate and other such like materials known in the art. Suitable buffers are generally selected to be chemically unreactive with the other ingredients that may be present in the solution, with the buffers present in amounts sufficient to provide the desired degree of pH buffering. Preferred pHs of the aqueous formulation of the present invention range from about 4.0 to about 5.0, such as for example about 4.5. Variations and adjustments of the pH of the aqueous formulation is preferably obtained by moderating the addition of the buffer salt(s).

Viscosity enhancing agents may be included as appropriate, for example in suspensions of the present invention, as determinable by one skilled in the art, to provide desired flow characteristic to the suspension. The amount of viscosity enhancing agent in the formulation is preferably sufficient to give a solution with a viscosity in the range of 10 to 100 centipoises, with a more preferred viscosity range of from about 20 to 90 centipoises, a still more preferred viscosity range of from about 25 to 75 centipoises, and a most preferred viscosity range of from about 50 to 60 centipoises. Representative viscosity enhancing agents suitable for inclusion in the present invention include, for example without limitation, Xanthan gum, sorbitol, glycerol, sucrose or cellulose derivatives in addition to the hydroxyethyl cellulose such as carboxymethylcellulose or a salt thereof of a C₁₋₄ alkyl and/or a hydroxy-C₂₋₄ alkyl ether of cellulose, such as methylcellulose, ethylcellulose, hydroxypropylcellulose, hydroxyethylmethylcellulose and hydroxypropylmethylcellulose.

Representative preservatives suitable for use in the present invention include, for example without limitation, one or more alkyl hydroxybenzoates, such as methyl hydroxybenzoates, ethyl hydroxybenzoates, propyl hydroxybenzoates, butyl hydroxybenzoates and the like. Additional preservatives useful in the present invention include, but are not limited to, sodium benzoate, potassium sorbate, and antimicrobial agents including parabens (p-hydroxybenzoic acids esters) such as methyl paraben, ethylparaben, propylparaben, butylparaben and the like, and combinations thereof. The preservatives listed herein are exemplary, with the appropriate preservative and amount of a preservative incorporated into the solution as determinable by one skilled in the art for compatibility and efficacy of the preservative in a given solution. Techniques and methods for evaluating preservative efficacy in a given pharmaceutical formulations are readily known in the art. Parabens are preferred, with methyl paraben most preferred for use as preservative ingredients to add to the present pharmaceutical solution, although other pharmaceutically acceptable preservatives may be substituted therefor. Preservatives may be included in a given pharmaceutical formulation of the present invention as appropriate, with preferred amounts of up to 1 gram per 100 mL of the solution. More preferably the preservatives are included in amounts that range of from about 0.10 to about 0.75 grams per 100 mL of the solution, still more preferably from about 0.15 to about 0.5 grams per 100 mL of the solution, and most preferably from about 0.20 to about 0.4 grams per 100 mL of the solution.

Coloring agents also may be incorporated in the solution of the present invention as determined by one skilled in the art to be appropriate, for chemical compatibility with other ingredients in the solution and the like. Coloring agents are generally used to provide an appealing color to the solution. Suitable coloring agents for use in pharmaceutical solutions are well known in the art. Such compounds include, by way of example and without limitation, FD&C Red No. 3, FD&C Red No. 20, FD&C Yellow No. 6, FD&C Blue No. 2, D&C Green No. 5, D&C Orange No. 5, D&C Red No. 8, caramel, and iron oxide (black, red, yellow), other F.D. & C. dyes and natural coloring agents such as grape skin extract, beet red powder, beta-carotene, annato, carmine, turmeric, paprika, combinations thereof and other such materials known to those skilled in the art.

The pharmaceutical formulation of the present invention preferably contains flavoring agents (herein referred to also as “flavorants”), sweetening agents, and combinations thereof to mask the inherently bitter taste associated with hydrocodone or the second active agent, and thereby improving the palatability of the solution of the present invention. Flavorants are used to impart a pleasant flavor and often odor to a pharmaceutical preparation. Suitable flavoring agents include natural and artificial flavors, such as synthetic flavor oils and flavoring aromatics and/or natural oils, extracts from plants, leaves, flowers, fruits and so forth and combinations thereof. Representative suitable flavoring agents may be for example, without limitation, menthol, cinnamon, wintergreen, clove, bay, anise, eucalyptus, thyme, cedar leave, nutmeg, sage, bitter almonds and cassia, vanilla, artificial vanilla, chocolate, artificial chocolate, bubble gum, both natural and artificial fruit flavors, such as cherry flavor, grape flavor, orange flavor, strawberry flavor, lemon flavor, grapefruit flavor and “mint” flavors such as peppermint flavor and spearmint flavor, lime flavor, apple flavor, pear flavor, peach flavor, raspberry flavor, plum flavor, pineapple flavor, apricot flavor and so forth, including combinations of two or more thereof. Flavoring agents are generally provided as a minor component of the solution in amounts effective to provide a palatable flavor to the solution. The amount of flavoring agent may depend on a number of factors, including the desired organoleptic effect. The precise amount of sweetening and/or flavoring agent(s) depends on the properties of the agent(s) used, however generally in an amount that is sufficient to mask the bitter taste associated with hydrocodone or the second active agent as determinable by one skilled in the art. However, flavoring agents are generally present in the solution in amounts in the range of from about 0 grams to about 10 grams per 100 mL of the solution, with preferred amounts of from about 2 grams to about 5 grams per 100 mL. Sweeteners suitable for inclusion in the present invention may be determined by one skilled in the art including, for example without limitation, both natural and artificial sweeteners such as the representative sweetening agents of intense sweeteners such as sorbitol, sucrose, saccharins such as sodium saccharin, cyclamates such as sodium cyclamates, aspartame, sucralose, thaumatin, acesulfame K, and the like, and sugars such as monosaccharides, disaccharides and polysaccharides. Representative sugars useful in the present invention include, without limitation, xylose, ribose, glucose, mannose, galactose, fructose, dextrose, sucrose, maltose, partially hydrolyzed starch or corn syrup, and sugar alcohols such as sorbitol, xylitol, mannitol, glycerin, etc. and combination thereof. Presently preferred as a sugar sweetener is sorbitol. The amount of sugar sweetener used in the solution varies with the degree of sweetening desired for the particular formulation as determinable by one skilled in the art, with preferred amounts of sugar sweetener ranging from about 0 grams to about 100 grams sugar sweetener per 100 mL of the solution, more preferably from about 20 grams to about 95 grams per 100 mL of solution, still more preferably from 30 grams to about 90 grams sugar sweetener per 100 mL of the solution, and most preferably from about 40 grams to about 85 grams per 100 mL of solution. Sugar sweeteners may be replaced or augmented by water soluble artificial sweeteners, such as the suitable artificial sweeteners previously listed and mixtures thereof. The amount of artificial sweetener used in the solution may vary to provide an appropriate amount of sweetness to the solution as determinable by one skilled in the art, generally in amounts similar to those of sugar sweeteners described above. Mixtures of sweetening and/or flavoring agents are preferably used.

The present invention provides methods of treating a subject (e.g., mammal, particularly humans) comprising administering to a subject in need of such treatment a therapeutically effective amount of at least one active ingredient, formulation thereof, or unit dose forms thereof, each as described herein.

As used herein, the term “treatment”, or a derivative thereof, contemplates partial or complete inhibition of the stated disease state such as, for example, pain, when an active ingredient of the present invention is administered prophylactically or following the onset of the disease state for which such active ingredient of the present invention is administered. For the purposes of the present invention, “prophylaxis” refers to administration of the active ingredient(s) to a mammal to protect the mammal from any of the disorders set forth herein, as well as others. The typical active daily dose of the hydrocodone depends on various factors such as, for example, the individual requirement of each patient, the route of administration, and the disease. An attending physician may adjust the dosage rate based on these and other criteria if he or she so desires. As an example, a suitable oral dosage form may encompass from about 2 mg to about 60 mg total daily dose, typically administered in one single dose or equally divided doses. A more preferred range is from about 7.5 mg to about 30 mg total daily dose, and a most preferred range is from about 30 mg total daily dose. It should be appreciated that daily doses other than those described above may be administered to a subject, as appreciated by an attending physician. The stabilized aqueous pharmaceutical hydrocodone composition of the present invention may also be administered as a pediatric formulation in appropriate unit dosage form.

As previously described, the hydrocodone API may be used as a single active agent, or may be combined with other active agents, vitamins, minerals, dietary supplements, etc. The therapeutic compound(s) contained within the present device can be formulated as its pharmaceutically acceptable salts. As used herein, “pharmaceutically acceptable salts” refers to derivatives of the disclosed compounds wherein the therapeutic compound is modified by reacting it with an acid or base as needed to form an ionically bound pair. Examples of pharmaceutically acceptable salts include conventional non-toxic salts or the quaternary ammonium salts of the parent compound formed, for example, from non-toxic inorganic or organic acids. Suitable non-toxic salts include those derived from inorganic acids such as hydrochloric, hydrobromic, sulfuric, sulfonic, sulfamic, phosphoric, nitric and others known to those of ordinary skill in the art. The salts prepared from organic acids such as amino acids, acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, isethionic, and others known to those of ordinary skill in the art. The pharmaceutically acceptable salts of the present invention can be synthesized from the parent therapeutic compound which contains a basic or acidic moiety by conventional chemical methods. Lists of other suitable salts are found in Remington's Pharmaceutical Sciences, 17^{th} ed., Mack Publishing Company, Easton, Pa., 1985, p. 1418, the relevant disclosure of which is hereby incorporated by reference.

The phrase “pharmaceutically acceptable” is employed herein to refer to those compounds, materials, compositions, and/or dosage forms which are, within the scope of sound medical judgment, suitable for use in contact with tissues of human beings and animals and without excessive toxicity, irritation, allergic response, or any other problem or complication, commensurate with a reasonable benefit/risk ratio.

The amount of therapeutic compound incorporated in each device of the invention will be at least one or more dosage forms and can be selected according to known principles of pharmacy. An effective amount of therapeutic compound is specifically contemplated. By the term “effective amount”, it is understood that, with respect to, for example, pharmaceuticals, a pharmaceutically effective amount is contemplated. A pharmaceutically effective amount is the amount or quantity of a drug or pharmaceutically active substance which is enough for the required or desired therapeutic response, or in other words, the amount which is sufficient to elicit an appreciable biological response when administered to a patient. The appreciable biological response may occur as a result of administration of single or multiple unit doses of an active substance. Depending upon the active substance used and upon the amount of active substance present in a particular device according to the invention, a unit dose may comprise one or more such devices.

Various formulations of the stabilized aqueous pharmaceutical hydrocodone composition of the present invention are illustrated in the examples below. As used herein, w/v represents weight per volume measurement and v/v represents volume per volume measurement.

### EXAMPLE 1 (Comparative)

A hydrocodone formulation of 0.5 grams of hydrocodone bitartrate and 33.4 grams of acetaminophen was formulated in a 1 liter batch. Under accelerated conditions of 40° C. at 75% relative humidity (40° C./75% RH) the hydrocodone decreased from 104.5% to 98.8%. In a second run, the hydrocodone decreased from 104.3% to 98.4%.

### EXAMPLE 2

A hydrocodone formulation of 0.5 grams of hydrocodone bitartrate, 0.5 grams of EDTA and 33.4 grams of acetaminophen were formulated in a 1 liter batch. Under accelerated conditions of 40° C. at 75% relative humidity (40° C./75% RH) the hydrocodone decreased from 106.6% to 106.2%.

### EXAMPLE 3

A hydrocodone formulation of 0.5 grams of hydrocodone bitartrate, 0.5 grams of EDTA and 33.4 grams of acetaminophen were formulated in a 1 liter batch. Under accelerated conditions of 40° C. at 75% relative humidity (40° C./75% RH) the hydrocodone decreased from 104.6% to 104.2%.

The foregoing summary, description, and examples of the invention are not intended to be limiting, but are only exemplary of the inventive features which are defined in the claims.

What is claimed is:

1. A stabilized hydrocodone pharmaceutical formulation, comprising:
a pharmaceutically effective amount of hydrocodone; and,
a stabilizing effective amount of ethylenediaminetetraacetic acid for stabilizing the hydrocodone.

2. The formulation of claim 1, wherein the pharmaceutical formulation comprises an oral liquid composition.

3. The formulation of claim 2, wherein the oral liquid composition is selected from the group consisting of pharmaceutically acceptable emulsions, solutions, suspensions, syrups, elixirs and combinations thereof.

4. The formulation of claim 3, wherein the oral liquid composition comprises a solution.

5. The formulation of claim 1, wherein the hydrocodone is present in an amount of from about 2 mg to about 12 mg per 15 mL.

6. The formulation of claim 5, wherein the hydrocodone is present in an amount of from about 7.5 mg per 15 mL.

7. The formulation of claim 1, wherein the ethylenediaminetetraacetic acid comprises an ethylenediaminetetraacetic salt.

8. The formulation of claim 1, wherein the ethylenediaminetetraacetic acid is present an amount of from about 0.01% w/v to about 0.2% w/v.

9. The formulation of claim 8, wherein the ethylenediaminetetraacetic acid is present an amount of from about 0.03% w/v to about 0.1% w/v.

10. The formulation of claim 7, wherein the ethylenediaminetetraacetic salt is selected from the group consisting of di-potassium salt, di-sodium salt, calcium/di-sodium salt, sodium salt, tri-sodium salt, tetrasodium salt and combinations thereof.

11. The formulation of claim 1, further comprising a pharmaceutically effective amount of a second active agent.

12. The formulation of claim 11, wherein the second active agent includes acetaminophen.

13. The formulation of claim 12, wherein the acetaminophen is present in an amount of from about 125 mg to about 750 mg per 15 mL.

14. The formulation of claim 13, wherein the acetaminophen is present in an amount of about 500 mg per 15 mL.

15. A unit dosage of the formulation of claim 1, wherein the hydrocodone is present in an amount of about 7.5 mg, and the ethylenediaminetetraacefic acid is present in an amount of about 0.05 w/v, per 15 mL of solution.

16. A unit dosage of the formulation of claim 12, wherein the acetaminophen is present in an amount of about 500 mg, the hydrocodone is present in an amount of about 7.5 mg, and the ethylenediaminetetraacetic acid is present in an amount of about 0.05 w/v, per 15 mL of solution.

17. A method for stabilizing a hydrocodone formulation, comprising the steps of:
formulating a hydrocodone composition; and,
adding to the formulated hydrocodone composition an effectively stabilizing amount of ethylenediaminetetraacetic acid.

18. The method of claim 17, further comprising the step of adding a second active agent to the hydrocodone composition.

19. The method of claim 18, wherein the second active agent comprises acetaminophen.

20. The method of claim 19, further comprising the step of formulating an oral liquid formulation.
